(19) United States
(12) Patent Application Publication
(10) Pub. No.: US 2005/0054927 A1
(43) Pub. Date: Mar. 10, 2005

(54) System and method for using scheduled protocol codes to automatically configure ultrasound imaging systems

(76) Inventor: Scott Love, Bothell, WA (US)

Correspondence Address: ATL ULTRASOUND, P.O. BOX 3003, 22100 BOTHELL EVERETT HIGHWAY, BOTHELL, WA, 98041-3003, US

(21) Appl. No.: 10/913,104

(22) Filed: Aug. 5, 2004

Related U.S. Application Data
(60) Provisional application No. 60/501,852, filed on Sep. 10, 2003.

(51) Int. Cl. A61B 8/00

(52) U.S. Cl. 600/443

(57) ABSTRACT

An ultrasound imaging system (10) allows the entry of protocol codes that correspond to respective ultrasound examinations, and then uses the protocol codes to automatically configure the imaging system (10) for the corresponding examination. The imaging system (10) uses the protocol codes to select all of the operating parameters for the imaging system (10), to prompt a sonographer using the imaging system (10) to attach the appropriate ultrasound probe (20), and to determine the content and format of a display (16) that is presented or a report that is generated at the conclusion of the examination.

This invention claims the benefit of Provisional U.S. Patent Application Ser. No. 60/501,852, filed Sep. 10, 2003.

### TECHNICAL FIELD

This invention relates to ultrasound imaging systems, and, more particularly, to a system and method facilitating the setup of ultrasound imaging systems based on the type of ultrasound examination that is to be performed.

### BACKGROUND OF THE INVENTION

Ultrasound imaging systems are widely used to obtain a variety of ultrasound images. Ultrasound imaging systems may be used to scan different parts of the body and the same parts of the body using different techniques or imaging modalities. For example, the arm of a patient may be scanned by placing an ultrasound transducer against different surfaces of the arm to obtain images from different directions. Further, each image may be obtained by either keeping the ultrasound transducer stationery or scanning the transducer across the surface of the skin while the image is being obtained. To obtain the proper image, the sonographer conducting the ultrasound examination must be provided with information indicating the type of ultrasound examination to be performed. The imaging system then must be configured in accordance with that information. Configuring the imaging system involves, for example, selecting the frequency of the transmitted and received ultrasound, selecting the imaging mode, such as frequency compounding, harmonic imaging, etc., selecting the type of scanhead to be used, and, in some cases, selecting the type of report that is to be generated from the examination.

Several techniques are conventionally used to configure ultrasound imaging systems. The most basic technique is for the sonographer conducting the ultrasound examination to simply read the necessary information from a patient's chart and then configure the imaging system for the examination procedure that is to be performed. Most hospitals and other patient care facilities use protocol codes to identify the type of ultrasound examination that is scheduled for a patient. For example, there may be one protocol code for an echo stress exam, another protocol code for an obstetrics ultrasound exam, another protocol code for a gastro-intestinal ultrasound exam, and so forth. The sonographer reads the protocol code from the patient's chart and configures the ultrasound imaging system accordingly based on either recollection or with references to a handbook or other document. The sonographer also generally enters patient identifying information, such as the patient's name or identification number, so that the identifying information can be displayed in a print-out or recording of the image.

There are several disadvantages and problems with the above-described technique for configuring ultrasound imaging systems. First, it requires a substantial period of time for the sonographer to read the chart, determine the protocol code(s) for one or more ultrasound examinations, and then configure the imaging system by manipulating controls or manually entering information and selecting a scanhead that is appropriate for the examination that is to be conducted. In the event the sonographer has not memorized the configuration parameters for each protocol code, the need to refer to a manual or other document further slows down the configuration of the imaging system. Second, this technique is prone to errors because it is fairly easy for an sonographer to incorrectly configure the imaging system for the examination that is to be performed, particularly if the sonographer is attempting to rely on memory for the configuration parameters corresponding to a protocol code. If the imaging system is configured incorrectly, the quality of the examination may very well be compromised.

Attempts have been made to solve the above-described productivity and error problems. One approach is to interface an ultrasound imaging system with a clinical information system that is maintained by many health-care providers. The clinical information system stores information about the patient, the procedures that are to be performed on the patient, information about physicians responsible for the patient, the patient's medical history, insurance information, and other information pertaining to the patient. The ultrasound imaging system may interface with the clinical information system through various means, such as a local area network or a wireless communication system. In use, the sonographer obtains patient identifying information from the patient or the patient's chart, and enters that information into the ultrasound imaging system. The ultrasound imaging system then transmits the patient identifying information to the clinical information system, which uses the patient identifying information to access information about the patient. The clinical information system then downloads a “digital requisition” to the ultrasound imaging system. The digital requisition includes information specific to the patient, such as the procedures that are to be formed, the name of the patient's physicians, insurance coverage information, medical alerts (HIV status, allergies, etc.) and other information about the patient. The digital requisition may also include information about the patient's medical history, including prior ultrasound images, which can be compared to the image being obtained during the examination procedure. The sonographer then configures the imaging system based on the digital requisition.

Although interfacing ultrasound imaging systems to clinical information systems provides significant performance advantages and lessens the possibility of mistakes, it is still less than ideal. It is still possible for the sonographer to enter the wrong patient identifying information, and thereby receive the wrong digital requisition. Also, it still requires significant time for the sonographer to properly configure the imaging system, and the sonographer may configure the imaging system incorrectly or less than optimum for the procedure that is to be performed.

A technique for ensuring that the correct digital requisition is used to configure an ultrasound imaging system is disclosed in U.S. Pat. No. 6,506,155 to Sluis. According to this approach, the patient to be examined is provided with some type of storage media, such as a bar code, smartcard, or personal digital assistant. The storage media stores patient identifying information that uniquely identifies the patient. The storage media is read by the ultrasound imaging system to determine the patient identifying information. The patient identifying information is then used to retrieve the digital requisition from either internal storage, such as a disk drive, or external storage, such a clinical information system. The retrieved digital requisition is then used to automatically configure the ultrasound imaging system. The technique described in the Sluis patent largely avoids the problem of incorrectly configuring the ultrasound imaging system for a patient as long as the patient provides the sonographer with storage media containing the correct patient identifying information. However, this technique requires that all patients that are to undergo an ultrasound examination be provided with storage media containing the correct patient identifying information. Also, the technique disclosed in the Sluis patent requires the sonographer to properly select an examination from a menu based on the digital requisition.

Another approach to facilitating the use of medical diagnostic systems is described in U.S. Pat. No. 5,361,755 to Schraag et al. The Schraag et al. system provides an instruction manual for operating a medical monitor. The instruction manual contains clear text instructions for setting up the monitor along with questions for the patient to answer. The instruction manual also includes respective bar codes corresponding to each answer. The patient configures the monitor in accordance with the instructions, and answers the questions by scanning the bar-code corresponding to the correct answer. The diagnostic information obtained by the monitor, as well as the patient's coded answers, are downloaded to a medical facility for analysis by a health-care practitioner. The codes may also be decoded by the monitor to provide clear text instructions for operating the monitor. Although the monitor described by Schraag et al. does facilitate the entry of information in to the monitor, the entered information does not automatically set up the monitor for any specific purpose nor does it tag the test results with information identifying the patient. As a result, the use of the Schraag et al. monitor is still time-consuming and prone to error.

There is therefore a need for a system that automatically configures ultrasound imaging systems in a manner that avoids the problems and limitations of conventional system and techniques for configuring ultrasound imaging systems.

### SUMMARY OF THE INVENTION

An ultrasound imaging system includes an ultrasound imaging probe coupled to an ultrasound signal path. The system also includes an output device for displaying or recording ultrasound images, and an input device for allowing a protocol code to be entered into the imaging system. These protocol codes are linked to respective sets of configuration parameters that may be accessed by the system. A processor included in the system determines the protocol code entered through the input device and then accesses the storage device to determine the set of configuration parameters corresponding to the entered protocol code. The processor then configures the ultrasound imaging system in accordance with the determined set of configuration parameters.

### BRIEF DESCRIPTION OF THE DRAWINGS

FIG. 1 is an isometric view an ultrasound imaging system in accordance with one embodiment of the present invention.

FIG. 2 is a block diagram of pertinent portions of the imaging system of FIG. 1.

FIG. 3 is a flowchart showing the software executed by a processor in the imaging system of FIG. 1 and showing the method in which the imaging system of FIG. 1 operates.

### DETAILED DESCRIPTION OF THE INVENTION

Embodiments of the present invention are directed to ultrasound imaging systems. Certain details are set forth below to provide a sufficient understanding of various embodiments of the invention. However, it will be clear to one skilled in the art that the invention may be practiced without these particular details. In other instances, well-known circuits, control signals, and timing protocols have not been shown in detail in order to avoid unnecessarily obscuring the invention.

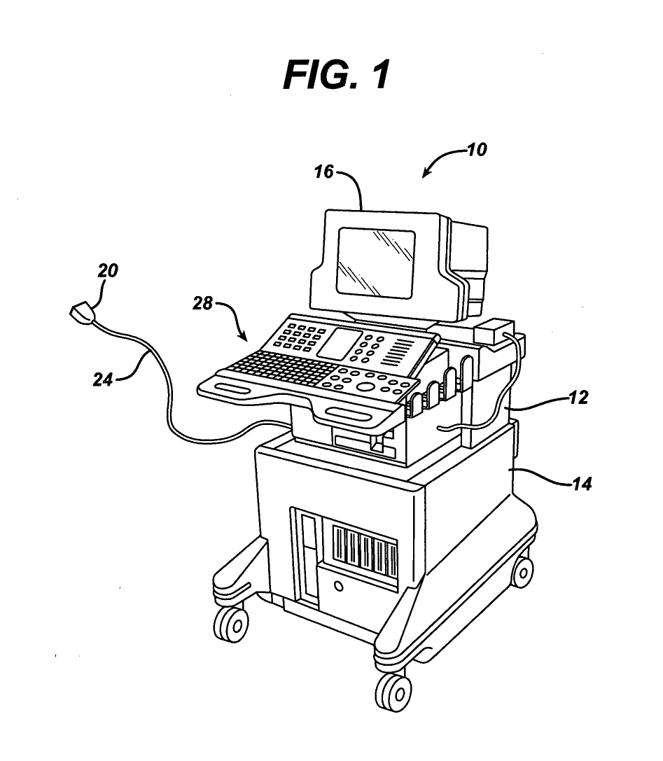

An ultrasound imaging system 10 in accordance with one embodiment of the invention is illustrated FIG. 1. The system 10 includes a chassis 12 containing most of the electronic circuitry for the system 10. The chassis 12 is mounted on a cart 14, and a display 16 is mounted on the chassis 12. An ultrasound imaging probe 20 is connected to the chassis 14 by a cable 24. Different imaging probes 20 are generally used for different types of ultrasound examinations. The chassis 12 includes a keyboard and controls, generally indicated by reference numeral 28, for allowing a sonographer to configure the imaging system 10 and enter information about the patient or the type of examination that is being conducted.

In operation, the probe 20 is placed against the skin of a patient (not shown) and either held stationery or moved to acquire an image of blood or tissues beneath the skin. The image is presented on the display 16, and it may be recorded by a recorder (not shown) or data storage medium (not shown in FIG. 1). The system 10 may also record or print a report containing text and images. Data corresponding to the image may also be downloaded through a suitable data link, such as the Internet or a local area network. The type of image shown on the display 16, the type of report recorded or printed, and the type of data downloaded will often depend on the type of ultrasound examination that is being conducted.

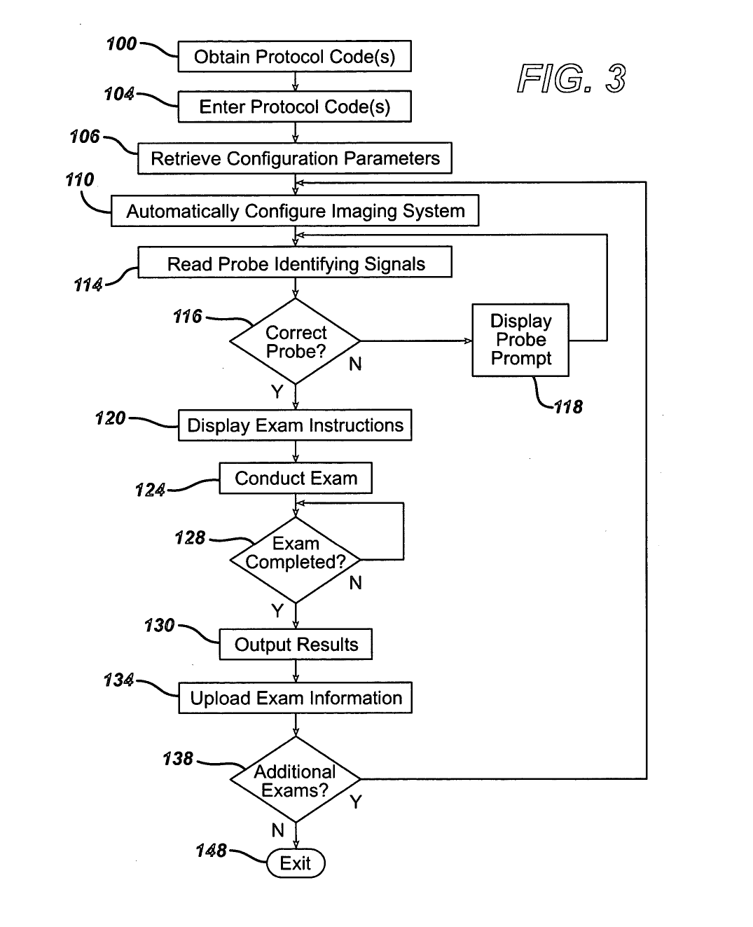

The above-described components of the imaging system 10 are conventional and are commonly used to obtain ultrasound images. The imaging system 10 according to one embodiment of the invention differs from conventional imaging systems by using protocol codes, which may be standardized throughout the healthcare field, to automatically configure the imaging system 10. The protocol codes are used in a manner that will be explained in detail in connection with FIG. 3.

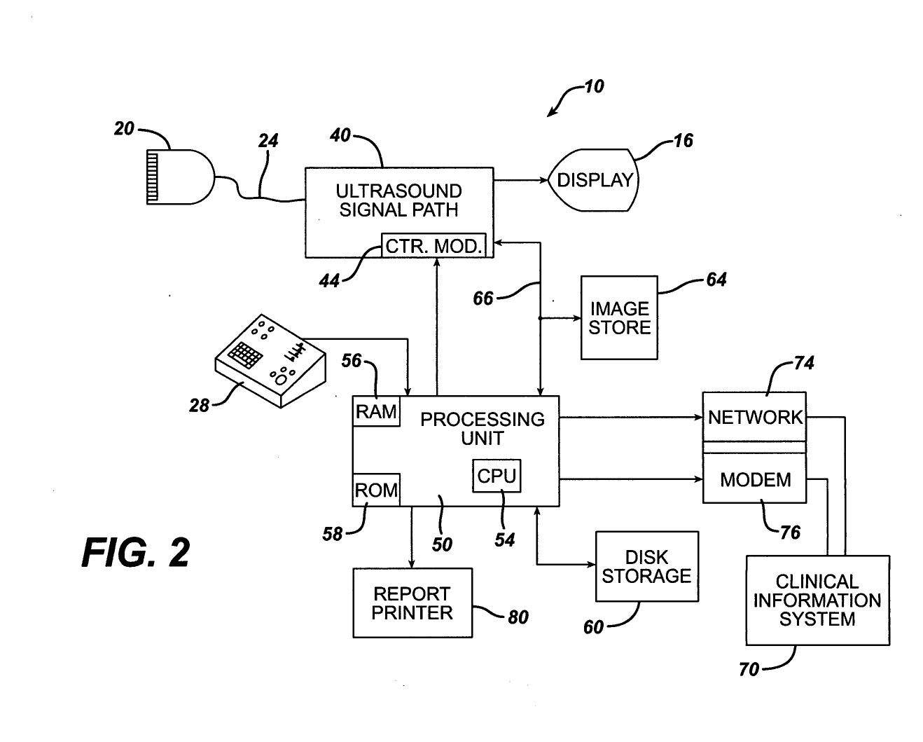

The electrical components in the ultrasound imaging system 10 are illustrated in greater detail in FIG. 2. The ultrasound imaging probe 20 is coupled through the cable 24 to an ultrasound signal path 40 of conventional design. Although one type of ultrasound imaging probe is shown in FIG. 2, it will be understood that other types of imaging probes can and generally will be used depending upon the type of ultrasound examination being conducted. In the embodiment shown in FIG. 2, the imaging probe 20 and all other imaging probes that will be used in the system 10 preferably provide probe identifying signals to a processing unit 50 to allow the processing unit 50 to determine the type of probe 20 currently being used.

As is well-known in the art, the ultrasound signal path 40 includes a transmitter (not shown) coupling electrical signals to the probe 20, an acquisition unit (not shown) that receives electrical signals from the probe 20 corresponding to ultrasound echoes, a signal processing unit (not shown) that processes the signals from the acquisition unit to perform a variety of functions, such as isolating returns from specific depths or isolating returns from blood flowing through vessels, and a scan converter (not shown) that converts the signals from the signal processing unit so that they are suitable for use by the display 16. The ultrasound signal path 40 also includes a control module 44 that interfaces with the processing unit 50 to control the operation of the above-described units. The ultrasound signal path 40 may, of course, contain components in addition to those described above, and, it suitable instances, some of the components described above may be omitted.

The processing unit 50 contains a number of components, including a central processor unit (“CPU”) 54, random access memory (“RAM”) 56, and read only memory (“ROM”) 58, to name a few. As is well-known in the art, the ROM 58 stores a program of instructions that are executed by the CPU 54, as well as initialization data for use by the CPU 54. The RAM 56 provides temporary storage of data and instructions for use by the CPU 54. The processing unit 50 interfaces with a mass storage device, such as a disk drive 60, for permanent storage of data, such as data corresponding to ultrasound images obtained by the system 10. However, such image data is initially stored in an image storage device 64 that is coupled to a signal path 66 extending between the ultrasound signal path 40 and the processing unit 50. The storage drive 60 also preferably stores sets of configuration parameters linked to respective protocol codes. However, in another embodiment the sets of configuration parameters linked to respective protocol codes are stored in the a clinical information system 70 that may be accessed through suitable means such as a local area network 74, a modem 76 or a wireless communication link (not shown). Therefore, once a protocol code has been entered into the system, the processing unit 50 can determine the set of configuration parameters that corresponds to the entered protocol code.

The processing unit 50 also interfaces with the keyboard and controls 28, which may be used to enter protocol codes. The keyboard and controls 28 may also be manipulated by the sonographer to manually configure the ultrasound imaging system and enter information. The processing unit 50 preferably interfaces with a report printer 80 that provides reports containing text and one or more images. The type of reports provided by the printer 80 preferably depends on the type of ultrasound examination that was conducted using the system 10.

The operation of the ultrasound imaging system 10 will now be explained with reference to FIG. 3. FIG. 3 comprises a flowchart showing the operation of the ultrasound imaging system 10, which is controlled by the processing unit 50 in accordance with a program stored in the ROM 58. The flowchart of FIG. 3 thus also constitutes an explanation of the software stored in the ROM 58 that is executed by the CPU 54.

The operation begins at step 100, where a sonographer reads a patient's chart to obtain the protocol code(s) for one or more ultrasound examinations that are to be conducted. However, the protocol code(s) may be obtained by means other than reading them from a patient's chart. The sonographer then enters the protocol code for the first (and possibly only) ultrasound examination that is to be performed at step 104. However, if more than one ultrasound examination is to be conducted, the sonographer can enter multiple protocol codes at step 104. The processing unit 50 retrieves the sets of configuration parameter(s) corresponding to the entered protocol code(s) at step 106. The processing unit 50 then uses the first set of configuration parameters to automatically configure the imaging system 10 in the optimum manner for the corresponding ultrasound examination at step 110. However, in other embodiments of the invention, the manner in which the imaging system is automatically configured at step 110 is determined by a combination of an entered protocol code and information about the patient, such as the patient's weight, age or sex.

Before the sonographer begins the ultrasound examination, the processing unit 50 reads the probe identifying signals from the probe 20 at step 114. The processing unit 50 then determines at step 116 if the probe connected to the ultrasound signal path 40 is appropriate for the examination that corresponds to the entered protocol code. If not, the processing unit 50 causes a message to be shown on the display 16 at step 118 that prompts the sonographer to connect the correct probe 20 to the ultrasound signal path 40. The operation then returns to step 114 to read the probe identifying signals and confirm at step 116 that the correct probe 20 is now connected to the ultrasound signal path 40.

Once the correct probe 20 is connected to the ultrasound signal path 40, the processing unit 40 causes an instructional message to be shown on the display 16 at step 120. The instruction message not only informs the sonographer that the system 10 is now correctly configured for the examination, but it also may provide some information about how the examination should be conducted. For example, the display 16 may provide the instruction “Scan Probe Along Skinline At A Constant Speed.” The sonographer then conducts the ultrasound examination at step 124. During this time, the processing unit waits at step 128 to determine when the examination has been completed, which is preferably signaled by the sonographer manipulating an appropriate key or control. The processing unit 50 then outputs the examination results at step 130 by causing the display 16 to display an image and/or cause the report printer 80 to provide a report and/or store the results in the disk drive 60. The examination results preferably includes text and at least one image, and the content and/or format of the examination results are a function of the protocol code entered at step 104. The processing unit 50 may also upload the examination results to the clinical information system 70 at step 134. Again, the content of the uploaded examination results is preferably a function of the protocol code entered at step 104.

After the report has been printed at step 130 and data have been uploaded at step 134, the processing unit checks at step 138 to determine if more than one protocol code was entered at step 104. If so, the operation returns to step 110 to automatically configure the system for the ultrasound examination for the next protocol code that was entered at step 104. If there was only one protocol code entered or examinations have been conducted for all entered protocol codes, the process exits at 148.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An ultrasound imaging system (10), comprising:
an ultrasound imaging probe (12);
an ultrasound signal path (40) coupled to the ultrasound imaging probe (12);
an output device (16) for displaying or recording examination results;
an entry device (28) for allowing a protocol code to be entered into the system (10),
a storage device (60) storing a plurality of sets of ultrasound imaging configuration parameters that are linked to respective protocol codes; and
a processor (50) coupled to the signal path (40), the output device (16), the entry device (28) and the storage device (60), the processor (50) being operable to determine the protocol code entered into the entry device (28), to access the storage device (60) to determine the set of configuration parameters corresponding to the entered protocol code, to configure the ultrasound imaging system (10) in accordance with the determined set of configuration parameters, and to cause the output device (16) to provide examination results corresponding the signals from which an ultrasound image can be generated.

2. The ultrasound imaging system (10) of claim 1, wherein the storage device (60) storing the sets of ultrasound imaging configuration parameters comprises a mass storage device included in the ultrasound imager.

3. The ultrasound imaging system (10) of claim 2, wherein the mass storage device (60) included in the ultrasound imager comprises a disk drive included in the ultrasound imaging system (10).

4. The ultrasound imaging system (10) of claim 1 wherein a plurality of different types of the ultrasound imaging probe (20) may be used in the ultrasound imaging system (10), and wherein the set of configuration parameters for at least some of the protocol codes includes information identifying the type of ultrasound imaging probe (20) that should be used to perform an ultrasound examination corresponding to the entered protocol code.

5. The ultrasound imaging system (10) of claim 4 wherein the processor (50) is further operable to display a message on the output device (16) prompting for the use of the type of ultrasound imaging probe (20) identified in the set of configuration parameters corresponding to the entered protocol code.

6. The ultrasound imaging system (10) of claim 4 wherein the output device (16) comprises a report generator coupled to the processor (50) for producing a report of an ultrasound examination, and wherein the set of configuration parameters for at least some of the protocol codes includes information identifying the content and format of the report, the processor (50) being operable to cause the report generator to generate a report corresponding to the entered protocol code.

7. The ultrasound imaging system (10) of claim 6 wherein the report generator comprises a report printer (80).

8. The ultrasound imaging system (10) of claim 1 wherein the sets of configuration parameters for at least some of the protocol codes includes information identifying the manner in which the ultrasound imaging probe (20) is used to conduct an ultrasound examination, and wherein the processor (50) is further operable to cause the output device (16) to display a message providing instructions on the manner in which the ultrasound imaging probe (20) is used to conduct an ultrasound examination corresponding to the entered protocol code.

9. The ultrasound imaging system (10) of claim 1 wherein the storage device (60) storing the sets of configuration parameters comprises a clinical information system (70) that is physically separate from the remaining components of the ultrasound imaging system (10).

10. The ultrasound imaging system (10) of claim 9, further comprising a communication link (not shown) coupled to the clinical information system (70) for allowing a set of configuration parameters linked to an entered protocol code to be retrieved.

11. The ultrasound imaging system (10) of claim 10 wherein the communication link comprises a modem (76).

12. The ultrasound imaging system (10) of claim 10 wherein the communication link comprises a local area network (74).

13. The ultrasound imaging system (10) of claim 1 wherein the output device comprises a display device (16) for displaying ultrasound images.

14. The ultrasound imaging system (10) of claim 1 wherein the processor (50) is operable to configure the ultrasound imaging system (10) in accordance with a combination of the determined set of configuration parameters and information about a patient who is to be examined.

15. A method of configuring an ultrasound imaging system (10), the method comprising:
storing a plurality of sets of configuration parameters that are linked to respective protocol codes;
entering a protocol code corresponding to an ultrasound examination that is to be conducted;
using the entered protocol code to access the set of configuration parameters linked to the entered protocol code; and
using the accessed set of configuration parameters to automatically configure the ultrasound imaging system (10).

16. The method of claim 15 wherein the act of entering a protocol code corresponding to an ultrasound examination that is to be conducted comprises manually entering the protocol into a data entry device (28) that is included in the ultrasound imaging system (10).

17. The method of claim 15 wherein the act of storing a plurality of sets of configuration parameters that are linked to respective protocol codes comprises storing the sets of configuration parameters in a mass storage device (60) that is physically a part of the ultrasound imaging system (10).

18. The method of claim 15 wherein at least some of the stored sets of configuration parameters include information identifying the type of ultrasound imaging probe (20) that should be used to perform an ultrasound examination corresponding to the entered protocol code.

19. The method of claim 18 wherein the act of using the accessed set of configuration parameters to automatically configure the ultrasound imaging system (10) comprises prompting for the use of the type of ultrasound imaging probe (20) identified in the set of the accessed set of configuration parameters.

20. The method of claim 15 wherein at least some of the stored sets of configuration parameters include information identifying the content and format of a report on an ultrasound examination generated by the ultrasound imaging system (10), and wherein the act of using the accessed set of configuration parameters to automatically configure the ultrasound imaging system (10) comprises using the accessed set of configuration parameters to generate a report containing the content and format identified by the accessed set of configuration parameters.

21. The method of claim 15 wherein at least some of the stored sets of configuration parameters include information identifying the manner in which the ultrasound imaging probe (20) is used to conduct an ultrasound examination, and wherein the act of using the accessed set of configuration parameters to automatically configure the ultrasound imaging system (10) comprises using the ultrasound imaging system (10) to automatically provide instructions on the manner in which the ultrasound imaging probe (20) is used to conduct an ultrasound examination corresponding to the entered protocol code.

22. The method of claim 15 wherein the act of storing a plurality of sets of configuration parameters that are linked to respective protocol codes comprises storing the sets of configuration parameters at a location remote from the ultrasound imaging system (10), and wherein the act of using the entered protocol code to access the set of configuration parameters linked to the entered protocol code comprises accessing the set of configuration parameters through a communications link.

23. The method of claim 15 wherein the act of using the accessed set of configuration parameters to automatically configure the ultrasound imaging system (10) comprises using a combination of the accessed set of configuration parameters and information about patient who is to be examined to automatically configure the ultrasound imaging system (10).
